(19) United States
(12) Patent Application Publication
(10) Pub. No.: US 2022/0304893 A1
(43) Pub. Date: Sep. 29, 2022

(54) PROTECTION OF OXYGEN SENSITIVE COMPOUNDS

(71) Applicant: BELOTECA, INC., San Diego, CA (US)

(72) Inventors: Fred Defesche, San Diego, CA (US); Dorla Mirejovsky, San Diego, CA (US); Shanghui Sun, San Diego, CA (US)

(73) Assignee: BELOTECA, INC., San Diego, CA (US)

(21) Appl. No.: 17/618,472

(22) PCT Filed: Jun. 16, 2020
(86) PCT No.: PCT/US20/37835

§ 371 Date: Dec. 12, 2021

Related U.S. Application Data
(60) Provisional application No. 62/862,620, filed on Jun. 17, 2019.
(60) Provisional application No. 62/986,507, filed on Mar. 6, 2020.

(51) Int. Cl. A61J 1/14; A61K 31/4985; A61K 31/555; A61K 47/02; A61J 1/06; A61M 15/08; A61M 15/00; B65D 81/26; B65B 5/04; B65B 55/00; B65D 77/04

(52) U.S. Cl. CPC A61J 1/1468; A61K 31/4985; A61K 31/555; A61K 47/02; A61J 1/065; A61M 15/08; A61M 15/009; B65D 81/266; B65B 5/045; B65B 55/00; B65D 77/0406

(57) ABSTRACT

Provided is a packaging system for an oxygen-sensitive compound (a drug, such as DHE, or an excipient such as exametazime), the system including a container, a self-activated oxygen scavenger (including an iron-based or non-iron based) and an oxygen-impermeable enclosure (e.g., an aluminum pouch). Also provided is a method of producing an oxygen-sensitive compound.

### CROSS-REFERENCE TO RELATED APPLICATIONS

This application claims the benefit of U.S. Provisional Application No. 62/862620, filed Jun. 17, 2019 and U.S. Provisional Application No. 62/986507, filed Mar. 6, 2020. Both applications are incorporated by reference herein in their entirety.

### BACKGROUND OF THE INVENTION

### (1) Field of the Invention

The present application generally relates to packaging of oxygen sensitive compounds. More specifically, the application provides packaging systems and methods of manufacturing oxygen sensitive compounds.

### (2) Description of the Related Art

Currently, many oxygen sensitive products are commercially available in ampoules that do not contain any oxygen as the oxygen is consumed during the sealing process. For convenience of use, products filled in syringes or cartridges are preferred. However, even when a product is manufactured under a strict oxygen protection and with no oxygen headspace in the container, over the shelf-life of the product, oxygen penetrates through the syringe tip or through an oxygen permeable closure of a selected container.

Other packaging for oxygen sensitive products include those described in Larson, 2015; U.S. Pat. Nos. 6,688,468; 7,708,719; 8,679,068; 9,248,229; 9,522,222; 9,840,359; 9,994,382; 10,035,129; 10,035,640; 10,035,879; 10,065,784; and 10,076,603; and US Patent Publications 2002/0132359; 2006/0076536; 2007/0010632; 2007/0163917; 2008/0008848; 2011/0240511; and 2017/0304150.

The present invention provides packaging that is very effective in preventing oxygen exposure to oxygen-sensitive compounds. The invention packaging is particularly useful for compounds in syringes, cartridges, nasal spray bottles, or vials.

### BRIEF SUMMARY OF THE INVENTION

Provided herewith is a pharmaceutical packaging system for an oxygen-sensitive drug. The packaging system comprises

at least one dosage of the oxygen-sensitive drug;

a medicament container containing at least one dosage of the oxygen-sensitive drug;

a self-activated oxygen scavenger; and

an oxygen-impermeable enclosure enclosing the medicament container and the self-activated oxygen scavenger, where the oxygen-impermeable enclosure is sealed.

Also provided is a method of producing an oxygen-sensitive compound. The method comprises

manufacturing the compound;

inserting the compound into a container;

place the container into an oxygen impermeable enclosure with a self-activated oxygen scavenger; and

seal the oxygen impermeable enclosure.

### BRIEF DESCRIPTION OF THE SEVERAL VIEWS OF THE DRAWINGS

FIG. 1 shows the effect of a sealed and an unsealed aluminum pouch on the stability of a syringe filled with dihydroergotamine mesylate (DHE) injection.

FIG. 2 is a graph showing levels of RRT 0.89 in DHE Injection pouched with two different oxygen scavengers over 10 months when stored at 25° C.

FIG. 3 is a graph showing formation of primary oxygen degradant in DHE Injection unprotected from oxygen.

FIG. 4 is a graph showing formation of RRT 0.86 and RRT 0.89 DHE nasal spray after storage.

### DETAILED DESCRIPTION OF THE INVENTION

Provided herein is packaging systems for oxygen sensitive compounds, and methods of packaging an oxygen sensitive compound. These systems and methods are useful for preserving any oxygen sensitive compound, for example a food or food ingredient, a dye, or a dry or liquid chemical. These systems and methods are particularly useful for packaging oxygen sensitive drugs for long term (months) storage.

Thus, in some embodiments, a packaging system for an oxygen-sensitive compound is provided. The packaging system comprises

the oxygen-sensitive compound;

a container containing the oxygen-sensitive compound;

a self-activated oxygen scavenger; and

an oxygen-impermeable enclosure enclosing the container and the self-activated oxygen scavenger, wherein the oxygen-impermeable enclosure is sealed.

Any oxygen-sensitive compound now known or later discovered can be usefully protected from oxygen using the systems provided herein. Nonlimiting examples of classes of compounds that include oxygen-sensitive compounds are drugs, excipients, dyes, metals, organometallic compounds, enzymes, and small molecules.

The oxygen-sensitive compound can be in liquid or solid form (e.g., a pill, powder or lyophilized product), with or without additional ingredients, e.g., excipients.

In some embodiments, the oxygen-sensitive compound is a drug. In these embodiments, the container is a medicament container containing at least one dosage of the oxygen sensitive drug. The medicament container can be any container that is used to hold a drug. Non-limiting examples include a pill bottle, a syringe, a cartridge, a spray bottle (e.g., a nasal spray bottle) or a vial, for example a stoppered glass or plastic vial, e.g., stoppered with a rubber stopper.

In other embodiments, the oxygen-sensitive compound is an excipient, i.e., a buffer, stabilizer, preservative, etc. that is mixed with a drug or other active substance. A non-limiting example of an oxygen-sensitive excipient is tin chloride, for example as used in radiopharmaceuticals, e.g.,exametazime for injection.

Technetium (Tc 99m) exametazime is a radiopharmaceutical sold under the trade name Ceretec™. It is used in cerebral scintigraphy to detect altered regional cerebral perfusion in stroke and other cerebrovascular diseases. Exametazime for injection is combined with technetium to make the Tc 99m exametazime. Each vial of exametazime for injection contains a lyophilized mixture of 0.5 mg exametazime, 7.6 μg stannous chloride dihydrate (minimum stannous tin 0.6 μg, maximum total stannous and stannic tin 4.0 μg/vial) and 4.5 mg sodium chloride, sealed under nitrogen atmosphere with a rubber closure.

The oxidation state of tin chloride is crucial to the performance of the product. The minimum content of stannous tin (Sn²⁺) must be 0.6 μg/vial, and the maximum content of total stannous and stannic tin (Sn²⁺ and Sn⁴⁺) must not exceed 4.0 μg/vial. The maximum amount of total stannous and stannic tin of 4.0 microgram/vial corresponds to 7.6 μg of tin chloride dihydrate/vial when taking into the consideration the molecular weights of tin (118.7 g/mol) and tin chloride dihydrate (225.62 g/mol).

Tin(II) chloride is a reducing agent and has an essential function in assisting the formation of a lipophilic technetium ^{99m}Tc complex when technetium Tc99m pertechnetate is added to exametazime. The lipophilic technetium Tc99m complex is the active moiety that can cross the blood-brain barrier.

Even when the drug product is manufactured under a strict control to prevent any oxidation of tin (II) to tin (IV), the oxidation of tin (II) occurs during storage at the recommended storage conditions of 15°-25° C. (59°-77° F.). When level of tin (II) drops below the stated level of 0.6 μg/vial, the product fails the test the for radiolabeled purity and fails the USP requirements for Technetium Tc99m Exametazime Injection. By using the packaging systems described herein, this oxidation is forestalled, increasing the shelf life of exametazime for injection.

The systems provided herein are not narrowly limited to the use of any particular oxygen-impermeable enclosure, since any such enclosure is expected to be useful here. Nonlimiting examples include high oxygen barrier polyethylene films (Ayuso et al., 2017), metal-containing flexible or rigid containers, e.g., a bag or a box, containing a metal (e.g., tin coated steel or aluminum) that may be laminated with, e.g., plastic. Nonlimiting examples of useful plastics for such containers include acrylonitrile butadiene styrene copolymer, cellulose acetate, cellulose acetate butyrate, chlorinated polyvinyl chloride, ethylene chlorotrifluoroethylene copolymer, ethylene methyl acrylate copolymer, ethylene tetrafluoroethylene copolymer, ethylene vinyl alcohol copolymer, ethylene vinyl acetate copolymer, ethylene vinyl chloride copolymer, high-density polyethylene, low-density polyethylene, linear low-density polyethylene, medium-density polyethylene, methyl ethyl ketone, oriented polypropylene, polyacrylonitrile, polybutylene terephthalate, polycarbonate, polychlorotrifluoroethylene, polyethylene, polyethylene terephthalate, polymethyl methacrylate, polypropylene, polystyrene, polytetrafluoroethylene, polyurethane, polyvinyl acetate, polyvinyl alcohol. polyvinyl butyral, polyvinyl chloride, polyvinylidene chloride, polyvinylidene fluoride, vinyl acetate, vinyl acetate ethylene copolymer, vinylidene chloride, nylon, mylar, polyester and polyethylene (Maekawa and Elert, 2003). Any self-activated (also called “self-reacting”—see http://ageless.mgc-a.com/AGELESS%20brochure.pdf) oxygen scavenger known in the art, including iron-based or non-iron-based (Id.), would be expected to be effective in this packaging. Non-limiting examples include Mitsubishi AGELESS® ZPT-100MBC and Multisorb Oxygen Scavenger StabilOx® D-100-H75.

In some embodiments, the oxygen-impermeable enclosure is metal-containing. In some of these embodiments, the metal is aluminum, e.g., a plastic-coated aluminum pouch. The container can contain any number of separate aliquots of the oxygen-sensitive compound (e.g., dosages of an oxygen sensitive drug or excipient), for example, 1, 2, 3, 4, 5, 6, 7, 8, 9, 10, 12, 14, 20, 24, 28, 50, greater than 50, or any number in between. In some embodiments, the oxygen-impermeable enclosure comprises more than one dosage of an oxygen-sensitive drug or excipient, where each dosage is in a separate medicament container, and where each medicament container is separated from every other medicament container by an oxygen impermeable barrier.

Although the Examples below describe the development of the above packaging for dihydroergotamine mesylate (DHE), the packaging would be expected to provide oxygen protection for any oxygen-sensitive compound, including oxygen-sensitive drugs or excipients. Nonlimiting examples of oxygen sensitive drugs or excipients that could advantageously be packaged in the above packaging include apomorphine, catecholamine drugs such as dopamine, epinephrine, norepinephrine, dobutamine and other structurally related compounds, ergotamine tartrate, dihydroergotamine mesylate (DHE), ephedrine, pseudoephedrine, a radiopharmaceutical comprising tin chloride, for example in exametazime for injection, acetaminophen, vitamin A, vitamin B, a vitamin D derivative, L-cysteine, and L-tryptophan. In some embodiments the oxygen-sensitive drug is epinephrine, dihydroergotamine mesylate (DHE), exametazime for injection or apomorphine.

Also provided is a method of producing an oxygen-sensitive compound, the method comprising

manufacturing the compound;

inserting the compound into a container;

place the container into an oxygen impermeable enclosure with a self-activated oxygen scavenger; and

seal the oxygen impermeable enclosure.

As described with the packaging system above, in some embodiments the compound is manufactured under oxygen deprivation conditions; in other embodiments the insertion of the compound into a container is under oxygen deprivation conditions.

As discussed above in relation to the systems provided herein, any oxygen-sensitive compound now known or later discovered can be usefully protected from oxygen using the systems provided herein.

Also as discussed above, the oxygen-sensitive compound can be in liquid or solid form (e.g., a pill, powder or lyophilized product), with or without additional ingredients, e.g., excipients.

Additionally as described above, in some embodiments, the oxygen-sensitive compound is a drug or an excipient.

In various embodiments, the oxygen-impermeable enclosure is metal-containing, e.g., aluminum, e.g., an aluminum pouch, where the medicament container contains one or more dosages of the oxygen-sensitive drug, e.g., where the oxygen-impermeable enclosure comprises more than one dosage of the oxygen-sensitive drug, where each dosage is in a separate medicament container, and where each medicament container is separated from every other medicament container by an oxygen impermeable barrier, also as discussed above.

These methods are useful for protection of any oxygen sensitive compound, e.g., apomorphine, catecholamine drugs such as dopamine, epinephrine, norepinephrine, dobutamine and other structurally related compounds, ergotamine tartrate, dihydroergotamine mesylate (DHE), ephedrine, pseudoephedrine, exametazime for injection, acetaminophen, vitamin A, vitamin B, a vitamin D derivative, L-cysteine, and L-tryptophan. In some embodiments the oxygen-sensitive drug is epinephrine, dihydroergotamine mesylate (DHE), exametazime for injection or apomorphine.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLE 1

### Dihydroergotamine Mesylate (DHE) Packaging Studies

The commercial product dihydroergotamine mesylate (DHE) injection, USP, is commercially provided in 1 mL sterile glass ampoules for subcutaneous use. It is sealed in glass ampoules to prevent degradation of the product by oxygen, due to the extreme sensitivity of DHE to oxygen. We evaluated several different DHE packaging regimes to determine whether DHE could be stored in a syringe, ready for injection, to have an injectable DHE product that is easier to use than the current commercial products.

### A. Requirement for Oxygen Protection of Syringes

Syringes filled with DHE were stored at either 40° C. or 25° C. in either a beaker (open to air) or placed in Pyrex® bottles flushed with nitrogen before tightly closing the cap. Samples were tested for stability at various time points. For each time-point a separate Pyrex® bottle was used to prevent any bottle opening when pulling the samples for testing.

DHE Injection forms two major degradants caused either by heat or presence of oxygen, as identified by HPLC as RRT 0.86 (primary heat effect degradant) or RRT 0.89 (primary oxygen effect degradant). The test results for the 40° C. condition are shown in Table 1, while Table 2 presents the data for the 25° C. condition.

As shown in Table 1 and Table 2, there is a clear difference between the levels of the two degradants when stored at 40° C. vs. when stored at 25° C. Also, more of the RRT 0.89 oxygen effect degradant was formed in syringes stored in a beaker than in a Pyrex® bottle flushed with nitrogen prior to closing. However, oxygen protection in Pyrex® bottles was not adequate after 3 months. Further, after six months in the Pyrex® packaging at 25° C., even the oxygen effect degradant RRT 0.89 was detected. Testing of a reserve sample at 10 months confirmed the 6-month data. The results also suggest that penetration of oxygen into Pyrex® bottles varied among bottles. The effect of oxygen is also manifested by discoloration and loss of potency.

We conclude from this study that syringes containing DHE injection require controlled oxygen protection over the shelf-life of the product.

TABLE 1
Stability of Dihydroergotamine Mesylate Injection at 40° C.

| | | Time Points | | | | | | | | | |
|---|---|---|---|---|---|---|---|---|---|---|---|
| | | No O₂ Protection - Stored in a Beaker | | | | | O₂ Protection - Stored in Pyrex Bottle | | | | |
| 40° C. | | T = 0 | 1 Month | 2 Months | 3 Months | 6 Months | T = 0 | 1 Month | 2 Months | 3 Months | 6 Months |
| Appearance | | CCSNVP | CCSNVP | CCSNVP | CSYNVP | YSNVP | CCSNVP | CCSNVP | CCSNVP | CSYNVP | CSYNVP |
| Assay (HPLC), % LC | | 100.7 | 103.8 | 99.7 | 95.3 | 91.2 | 100.7 | 102.7 | 99.7 | 95.9 | 92.5 |
| Related | RRT | ND | 0.40 | 0.68 | 0.73 | 1.36 | ND | 0.36 | 0.59 | 1.00 | 1.65 |
| Substances | 0.86 | | | | | | | | | | |
| by HPLC, | RRT | 0.03 | 0.30 | 0.81 | 1.41 | 2.48 | 0.03 | ND | ND | 0.04 | ND |
| % w/w | 0.89 | | | | | | | | | | |

| | | | | | | | | | | |
|---|---|---|---|---|---|---|---|---|---|---|
| Total | 0.16 | 1.16 | 2.59 | 4.27 | 7.93 | 0.16 | 0.83 | 1.35 | 2.07 | 3.28 |

CCSNVP = Clear, colorless solution with no visible particulates
CSYNVP = Clear, slightly yellow solution with no visible particulates
YSNVP = Yellow solution with no visible particulates

TABLE 2
Stability of Dihydroergotamine Mesylate Injection at 25° C.

| | | Time Points | | | | | | | |
|---|---|---|---|---|---|---|---|---|---|
| | | No O₂ Protection - Stored in a Beaker | | | | O₂ Protection - Stored in Pyrex Bottle | | | |
| 25° C. | | T = 0 | 3 Months | 6 Months | ~10 Months | T = 0 | 3 Months | 6 Months | ~10 Months |
| Appearance | | CCSNVP | CCSNVP | YSNVP | YSNVP | CCSNVP | CCSNVP | CSYNVP | CSYNVP |
| Assay (HPLC), % LC | | 100.7 | 96.0 | 94.1 | 82.1 | 100.7 | 102.1 | 94.5 | 95.9 |
| Related | RRT | ND | 0.26 | 0.50 | 0.63 | ND | 0.06 | 0.42 | 0.26 |
| Substances | 0.86 | | | | | | | | |
| by HPLC, | RRT | 0.03 | 1.94 | 3.20 | 4.73 | 0.03 | 0.08 | 2.63 | 1.26 |
| % w/w | 0.89 | | | | | | | | |

| | | | | | | | | |
|---|---|---|---|---|---|---|---|---|
| Total | 0.16 | 3.9 | 5.8 | 8.3 | 0.16 | 0.25 | 4.9 | 2.6 |

ND = Not Detected
CCSNVP = Clear, colorless solution with no visible particulates
CSYNVP = Clear, slightly yellow solution with no visible particulates
YSNVP = Yellow solution with no visible particulates

### B. Aluminum Pouch

To evaluate the effect of aluminum pouches as a possible protection of DHE injection, syringes were placed in aluminum pouches and sealed. Half of the pouches were sealed under ambient conditions while the other half were not sealed as a control. The pouches were placed in 25° C. and 40° C. chambers and tested over a 6-month period.

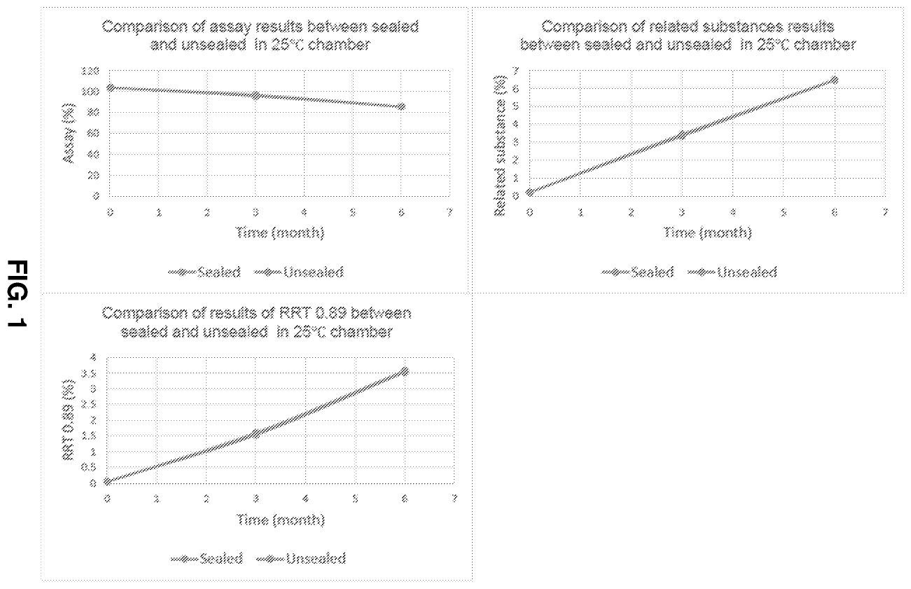

Results for the 25° C. storage condition are presented graphically in FIG. 1. The results show that the amounts of the oxygen degradant RRT 0.89, the total related impurities and the assay values were identical for both conditions. Those sealed aluminum pouches under ambient conditions did not provide any protection for DHE Injection filled in a syringe.

### C. Aluminum Pouch Nitrogen vs. Desiccant

Syringes filled with DHE injection were either pouched and sealed under nitrogen or placed in pouches under ambient oxygen conditions with a non-self-activated oxygen scavenger. The pouches were placed either in 25° C. and 40° C. chambers and tested over a period of 3 months. The amounts of the primary heat degradant RRT 0.86 and the primary oxygen degradant RRT 0.89, and the amounts of total related substances are presented in Table 3 for 25° C.-storage temperature.

The data shown in Table 3 indicate that sealing a pouch under nitrogen is not sufficient to prevent oxygen degradation. Similarly, the presence of non-self-activated oxygen scavenger has no effect on the degradation of DHE since the amounts of the primary oxygen degradant RRT 0.89 were very close for both conditions.

TABLE 3
Stability of Dihydroergotamine Mesylate Injection
at 25° C. - Effect of Nitrogen and Desiccant

| | | Time Points | | | | | |
|---|---|---|---|---|---|---|---|
| | | Pouched Under Nitrogen | | | Pouched with Desiccant | | |
| 25° C. | | T = 0 | 1 Month | 3 Months | T = 0 | 1 Month | 3 Months |
| Appearance | | CCSNVP | CCSNVP | CSYNVP | CCSNVP | CCSNVP | CSYNVP |
| Assay (HPLC), % LC | | 99.6 | 96.2 | 84.7 | 99.6 | 95.4 | 85.4 |
| Related | RRT | 0.16 | 0.21 | 0.41 | 0.16 | 0.24 | 0.45 |
| Substances | 0.86 | | | | | | |
| by HPLC, | RRT | 0.72 | 1.2 | 2.5 | 0.72 | 1.4 | 2.9 |
| % w/w | 0.89 | | | | | | |

| | | | | | | |
|---|---|---|---|---|---|---|
| Total | 1.7 | 2.8 | 5.4 | 1.7 | 3.3 | 6.2 |

CCSNVP = Clear, colorless solution with no visible particulates
CSYNVP = Clear, slightly yellow solution with no visible particulates

### D. Aluminum Pouch with Self-activated Oxygen Absorbing Materials

In the next set of experiments, syringes filled with DHE injection were placed in aluminum pouches with two types of self-activated oxygen absorbing materials, Oxygen Scavenger AGELESS° ZPT-100MBC manufactured by Mitsubishi Gas Chemical America, Inc. and Oxygen Scavenger StabilOx® D-100-H75 by MULTISORB Technologies. Each individual syringe was inserted in an aluminum pouch together with one of the oxygen scavengers prior to sealing the pouch. Pouches were placed on stability for 6 months.

The data shown in Table 4 demonstrate the efficacy of oxygen scavengers in preventing the degradation of DHE through oxidation.

The presence of oxygen scavengers was even more effective in stopping the degradation of samples previously exposed to oxygen for several months, as shown in Table 5. Syringes filled with DHE injection were left exposed to oxygen for 4 months, then pouched with oxygen scavengers and placed in stability chambers.

TABLE 4
Stability of Dihydroergotamine Mesylate Injection in Presence of Oxygen Scavengers at 25° C.

| | | Time Points | | | | | | | |
|---|---|---|---|---|---|---|---|---|---|
| | | | | | | StabilOx ® D-100-H75 by | | | |
| | | AGELESS ® ZPT-100MBC Mitsubishi | | | | MULTISORB Technologies. | | | |
| 25° C. | | T = 0 | 1 Month | 3 Months | 6 Months | T = 0 | 1 Month | 3 Months | 6 Months |
| Appearance | | CCSNVP | CCSNVP | CCSNVP | CCSNVP | CCSNVP | CCSNVP | CCSNVP | CCSNVP |
| Assay (HPLC), % LC | | 103.1 | 99.5 | 94.2 | 90.7 | 103.1 | 99.1 | 94.6 | 91.0 |
| Related | RRT | ND | 0.04 | 0.08 | 0.13 | ND | 0.04 | 0.08 | 0.13 |
| Substances | 0.86 | | | | | | | | |
| by HPLC, | RRT | 0.05 | 0.09 | 0.13 | 0.04 | 0.05 | 0.06 | 0.09 | 0.04 |
| % w/w | 0.89 | | | | | | | | |

| | | | | | | | | |
|---|---|---|---|---|---|---|---|---|
| Total | 0.23 | 0.30 | 0.43 | 0.40 | 0.23 | 0.26 | 0.37 | 0.43 |

ND = Not Detected
CCSNVP = Clear, colorless solution with no visible particulates

TABLE 5
Effect of Oxygen Scavengers on Previously Oxygen Exposed Samples

| | | Time Points | | | | | | | |
|---|---|---|---|---|---|---|---|---|---|
| | | | | | | StabilOx ® D-100-H75 by | | | |
| | | AGELESS ® ZPT-100MBC Mitsubishi | | | | MULTISORB Technologies. | | | |
| 25° C. | | T = 0 | 1 Month | 3 Months | 6 Months | T = 0 | 1 Month | 3 Months | 6 Months |
| Appearance | | CCSNVP | CSYNVP | YSNVP | YSNVP | CCSNVP | CSYNVP | YSNVP | YSNVP |
| Assay (HPLC), % LC | | 95.0 | 92.3 | 88.8 | 88.9 | 95.0 | 93.1 | 90.6 | 90.8 |
| Related | RRT | 0.21 | 0.28 | 0.25 | 0.31 | 0.21 | 0.29 | 0.45 | 0.31 |
| Substances | 0.86 | | | | | | | | |
| by HPLC, | RRT | 1.3 | 2.0 | 2.5 | 2.4 | 1.3 | 2.1 | 2.7 | 2.5 |
| % w/w | 0.89 | | | | | | | | |

| | | | | | | | | |
|---|---|---|---|---|---|---|---|---|
| Total | 2.8 | 4.2 | 4.7 | 4.40 | 2.8 | 4.3 | 5.5 | 4.9 |

CCSNVP = Clear, colorless solution with no visible particulates
CSYNVP = Clear, slightly yellow solution with no visible particulates
YSNVP = Yellow solution with no visible particulates

### EXAMPLE 2

### Additional Dihydroergotamine Mesylate Packaging Studies

Dihydroergotamine Mesylate (DHE) Injection, USP, filled in syringes was pouched with an oxygen scavenger. Two types of self-activated oxygen absorbing materials were investigated: Oxygen Scavenger AGELESS° ZPT-100MBC manufactured by Mitsubishi Gas Chemical America, Inc. and Oxygen Scavenger StabilOx® D-100-H75 by MULTISORB Technologies. Each individual syringe was inserted in a pouch together with one of the oxygen scavenger type prior to sealing the pouch. Levels of degradation were monitored over the period of 10 months when stored at 25° C. DHE forms one specific degradant with RRT 0.89 in the presence of oxygen.

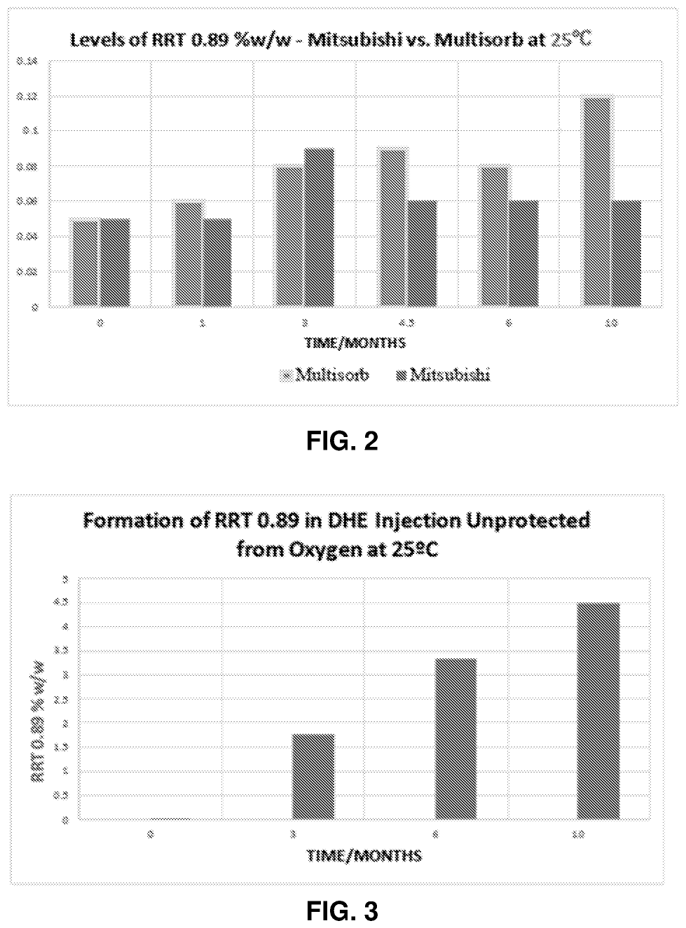

FIG. 2 shows that the levels of the primary oxygen degradant with RRT 0.89 remained more or less unchanged over a period of 10 months. This is in sharp contrast with the results depicted in FIG. 3 when the levels of the primary oxygen degradant reached about 4.5% when the syringes with DHE Injection were stored horizontally at 25° C. for 10 months without any oxygen protection.

The results shown in FIG. 2 demonstrate that oxygen scavengers in the aluminum pouch remained active over a period of 10 months and assured a good stability of the product.

To evaluate the oxygen scavenging capacity of scavengers after 10 months, the scavengers were removed from the remaining 10-month old samples and evaluated for their oxygen scavenging capacity. Each scavenger was placed in one 500-mL Pyrex bottle (with a total volume of 610 mL) for 12 days of storage at room temperature and then the oxygen level in each bottle was tested. The results are presented in Table 6. All scavengers maintained their oxygen scavenging capacity, StabilOx® D-100-H75 from Multisorb appeared to retain at 10 months higher oxygen scavenging capacity than AGELESS® ZPT-100MBC from Mitsubushi.

TABLE 6
Evaluation of Remaining Oxygen Scavenging
Capacity after 10 Months

| Sample | | Oxygen Level in a 500 mL |
|---|---|---|
| No. | Scavenger Type | Pyrex Bottle after 12 Days |
| 1 | StabilOx ® D-100-H75 - | 12.5% |
| | Multisorb | |
| 2 | StabilOx ® D-100-H75 - | 10.4% |
| | Multisorb | |
| 3 | AGELESS ® ZPT-100MBC | 17.1% |
| | Mitsubishi | |

These results demonstrate that degradation of oxygen sensitive products filled in syringes can be prevented by pouching the syringes with self-activated oxygen absorbing materials.

### EXAMPLE 3

### Oxygen Protection of Inhalation Spray Bottles

Effectiveness of oxygen scavengers in consuming the oxygen in the pouches and inside the nasal spray bottles was also evaluated. Packaging an oxygen sensitive compound like DHE in a pouch with an oxygen scavenger opens a new way to develop a nasal spray not requiring any assembly prior to use.

At the outset, the method to test the level of oxygen using Oxygen Analyzer, Quanteck Instruments, was verified. Three (3) aluminum mylar pouches were purged with nitrogen before sealing them. Another three (3) aluminum mylar pouches were inflating with air and sealed. Then, each pouch was punctured with the needle of Oxygen Analyzer's probe and the gas was squeezed out of the pouch to determine the oxygen level in each pouch. The results shown in Table 7 demonstrate the suitability of the technique to determine the oxygen levels in sealed pouches.

TABLE 7
Oxygen Determination in Aluminum Mylar Pouches

| | Tests | | | | | |
|---|---|---|---|---|---|---|
| | Pouch Filled with Air | | | Pouch Filled with Nitrogen | | |
| | Test 1 | Test 2 | Test 3 | Test 1 | Test 2 | Test 3 |
| Oxygen Level | 19.3% | 18.7% | 20.1% | 0.71% | 0.92% | 0.69% |

Next, the ability of oxygen scavengers to decrease the level of oxygen in a pouch and in a nasal spray plastic bottle was investigated.

One nasal spray plastic bottle with actuator and one oxygen scavenger were placed into an aluminum mylar pouch. The pouch was inflated with air and then sealed. Two types of scavengers were used: Mitsubishi Gas America, Inc. AGELESS ZPT-200MBC and Multisorb, Stabilox D-100-H75.

The oxygen level inside the aluminum mylar pouch and in a nasal spray plastic bottle were determined after 1, 3 and 7 days. The results are presented in Table 8. It can be seen that:
1. Mitsubishi AGELESS oxygen scavenger consumed oxygen faster than the Multisorb Stabilox oxygen scavenger.
2. Mitsubishi AGELESS oxygen scavenger consumed all oxygen in the pouch within 24 hours and all oxygen in the spray bottle within 3 days, while Multisorb Stabilox oxygen scavenger consumed around 95% of oxygen in the pouch and spray bottle within a week.

TABLE 8
Oxygen Levels in a Pouch and Nasal Spray Bottle

| | | AGELESS ZPT-200MBC | | Multisorb, Stabilox D-100-H75 | |
|---|---|---|---|---|---|
| | | Residual % | Residual % | Residual % | Residual % |
| | | Oxygen in | Oxygen in | Oxygen in | Oxygen in |
| Time | | Aluminum | Nasal Spray | Aluminum | Nasal Spray |
| Point | | Mylar Pouch | Bottle | Mylar Pouch | Bottle |
| 1 | Day | 0.30% | 9.53% | 10.80% | 16.18% |
| 3 | Days | 0.36% | 0.72% | 6.60% | 7.33% |
| 7 | Days | 0.35% | 0.68% | 1.04% | 1.47% |

The next experiment investigated the effectiveness of a scavenger to decrease the oxygen level in a spray bottle assembled as an engine and nozzle.

A nasal spray was assembled using 3 components: a nozzle, an engine, and a bottle (either amber glass bottle or white plastic bottle). Each nasal spray bottle was placed in an aluminum mylar pouch with one Mitsubishi scavenger (AGELESS ZPT-200MBC), the pouch was inflated with air and then sealed.

The oxygen levels were tested after 1, 3 and 7 days as described above. Table 9 shows the results confirming the previous findings:
1. Mitsubishi AGELESS oxygen scavenger can consume all oxygen in the pouch within 24 hours and all oxygen in the spray bottles within 3 days.
2. Significant reduction in oxygen level was observed between day 1 and 3 for both amber and white plastic spray bottle configurations.
3. Oxygen levels within the pouch were consistent throughout the study.
4. After day 3, no significant drop in the oxygen level could be seen, the oxygen levels remained constant.

TABLE 9
Oxygen Levels in Nasal Spray Bottles
Pouched with Mitsubishi Scavenger

| | | Amber Nasal Spray Bottle | | White Nasal Plastic Spray Bottle | |
|---|---|---|---|---|---|
| | | Residual % | Residual % | Residual % | Residual % |
| | | Oxygen in | Oxygen in | Oxygen in | Oxygen in |
| Time | | Aluminum | Nasal Spray | Aluminum | Nasal Spray |
| Point | | Mylar Pouch | Bottle | Mylar Pouch | Bottle |
| 1 | Day | 0.28% | 1.63% | 0.23% | 1.78% |
| 3 | Days | 0.30% | 0.41% | 0.21% | 0.53% |
| 7 | Days | 0.20% | 0.46% | 0.25% | 0.45% |

In conclusion, it is clear that oxygen sensitive compounds can be developed as a nasal spray by pouching the bottle with self-activated oxygen absorbing materials.

### EXAMPLE 4

### Oxygen Protection of Inhalation Spray Bottles Stability Study

To demonstrate the effectiveness of oxygen scavengers to protect DHE in Dihydroergotamine Mesylate Nasal Spray, 4 mg/mL, 2 mL of DHE Inhalation solution were dispensed in Nemera 10 mL White HDPE Plastic Bottles assembled with Nemera engine and nozzle and pouched with Mitsubishi oxygen scavenger AGELESS ZPT-200MBC. The pouches were place in stability chambers at 40° C.

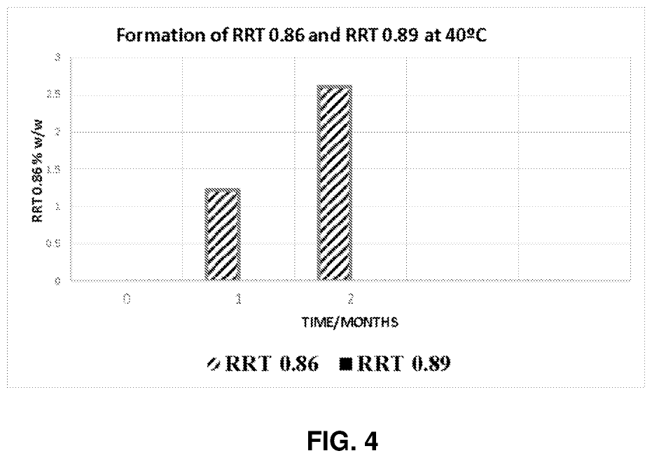

The results shown in FIG. 4 demonstrate that oxygen scavengers in the aluminum pouch were effective to eliminate the formation of oxygen sensitive related substance RRT 0.89. None was detected after a storage of 2 months at 40° C. The sole degradant detected is RRT 0.86 formed by heat.

### REFERENCES

Ageless® Oxygen Absorber Brochure, at http://ageless.mgc-a.com/AGELESS%20brochure.pdf.

Ayuso et al., 2017, Polymers & Polymer Composites, 25:571-582.

Larson, 2015, at https://www.healthcarepackaging.com/article/package-component/desiccants/oxygen-absorption-takes-many-forms-packages.

Maekawa and Elert, 2003, Chapters 2 and 3 in The Use of Oxygen-Free Environments in the Control of Museum Insect Pests, The Getty Conservation Institute.

U.S. Pat. No. 6,688,468

U.S. Pat. No. 7,708,719

U.S. Pat. No. 8,679,068

U.S. Pat. No. 9,248,229

U.S. Pat. No. 9,522,222

U.S. Pat. No. 9,840,359

U.S. Pat. No. 9,994,382

U.S. Pat. No. 10,035,129

U.S. Pat. No. 10,035,640

U.S. Pat. No. 10,035,879

U.S. Pat. No. 10,065,784

U.S. Pat. No. 10,076,603

US Patent Publication 2002/0132359

US Patent Publication 2006/0076536

US Patent Publication 2007/0010632

US Patent Publication 2007/0163917

US Patent Publication 2008/0008848

US Patent Publication 2011/0240511

US Patent Publication 2017/0304150

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

What is claimed is:

1. A packaging system for an oxygen-sensitive compound, the packaging system comprising
the oxygen-sensitive compound;
a container containing the oxygen-sensitive compound;
a self-activated oxygen scavenger; and
an oxygen-impermeable enclosure enclosing the container and the self-activated oxygen scavenger, wherein the oxygen-impermeable enclosure is sealed.

2. The packaging system of claim 1, wherein the oxygen-sensitive compound is a drug or an excipient for a drug.

3. The packaging system of claim 2, wherein the container is a syringe.

4. The packaging system of claim 2, wherein the container is a cartridge.

5. The packaging system of claim 2, wherein the container is a nasal spray bottle.

6. The packaging system of claim 2, wherein the container is a vial

7. The packaging system of claim 6, wherein the vial is a stoppered glass vial.

8. The packaging system of claim 1, wherein the oxygen-impermeable enclosure is metal-containing.

9. The packaging system of claim 8, wherein the metal is aluminum.

10. The packaging system of claim 1, wherein the oxygen-impermeable enclosure is an aluminum pouch.

11. The packaging system of claim 2, wherein the container contains one dosage of an oxygen-sensitive drug.

12. The pharmaceutical packaging system of claim 2, wherein the container contains more than one dosage of an oxygen-sensitive drug.

13. The packaging system of claim 12, wherein each dosage is in a separate container, wherein each container is separated from every other medicament container by an oxygen impermeable barrier.

14. The packaging system of claim 2, wherein the oxygen-sensitive compound is apomorphine, dopamine, epinephrine, norepinephrine, ergotamine tartrate, dihydroergotamine mesylate (DHE), ephedrine, pseudoephedrine, acetaminophen, exametazime for injection, a radiopharmaceutical comprising tin chloride, tin chloride in exametazime for injection, vitamin A, vitamin B, a vitamin D derivative, L-cysteine, or L-tryptophan.

15. The packaging system of claim 2, wherein the oxygen-sensitive compound is epinephrine, dihydroergotamine mesylate (DHE), exametazime for injection or apomorphine.

16. The packaging system of claim 2, wherein the oxygen-sensitive compound is dihydroergotamine mesylate (DHE).

17. The packaging system of claim 2, wherein the oxygen-sensitive compound is exametazime for injection.

18. The packaging system of claim 2, wherein the oxygen-sensitive compound is an excipient comprising tin (II).

19. The packaging system of claim 18, wherein the excipient is useful for combining with a radioactive diagnostic agent.

20. The packaging system of claim 19, wherein the excipient is exametazime for injection.

21. A method of producing an oxygen-sensitive compound;
inserting the compound into a container;
place the container into an oxygen impermeable enclosure with a self-activated oxygen scavenger; and
seal the oxygen impermeable enclosure.

22. The method of claim 21, wherein the manufacturing and/or the inserting is under oxygen-sensitive conditions.

23. The method of claim 21, wherein the oxygen-sensitive compound is a drug or an excipient.

24. The method of claim 21, wherein the container is a syringe.

25. The method of claim 21, wherein the container is a cartridge.

26. The method of claim 21, wherein the container is a nasal spray bottle.

27. The method of claim 21, wherein the container is a vial.

28. The method of claim 27, wherein the vial is a stoppered glass vial.

29. The method of claim 21, wherein the oxygen-impermeable enclosure is metal-containing.

30. The method of claim 29, wherein the metal is aluminum.

31. The method of claim 21, wherein the oxygen-impermeable enclosure is an aluminum pouch.

32. The method of claim 23, wherein the container contains one dosage of the oxygen-sensitive drug or excipient.

33. The method of claim 23, wherein the container contains more than one dosage of the oxygen-sensitive drug or excipient.

34. The method of claim 23, wherein the oxygen-impermeable enclosure comprises more than one dosage of the oxygen-sensitive drug or excipient, wherein each dosage is in a separate medicament container, wherein each medicament container is separated from every other medicament container by an oxygen impermeable barrier.

35. The method of claim 23, wherein the oxygen-sensitive compound is apomorphine, dopamine, epinephrine, norepinephrine, ergotamine tartrate, dihydroergotamine mesylate (DHE), ephedrine, pseudoephedrine, acetaminophen, a radiopharmaceutical comprising tin chloride, tin chloride in exametazime for injection, vitamin A, vitamin B, a vitamin D derivative, L-cysteine, or L-tryptophan.

36. The method of claim 23, wherein the oxygen-sensitive compound is epinephrine, dihydroergotamine mesylate (DHE), exametazime for injection or apomorphine.

37. The method of claim 23, wherein the oxygen-sensitive compound is dihydroergotamine mesylate (DHE) or exametazime for injection.
